(19) United States
(12) Patent Application Publication
(10) Pub. No.: US 2024/0216038 A1
(43) Pub. Date: Jul. 4, 2024

(54) APPLYING PULSED ELECTRIC FIELDS IN THE TREATMENT OF THE VASCULATURE

(71) Applicant: Galvanize Therapeutics, Inc., Redwood City, CA (US)

(72) Inventors: Steven D. GIROUARD, Chagrin Falls, OH (US); Jonathan R. WALDSTREICHER, West Orange, NJ (US); Robert E. Neal, II, Redwood City, CA (US)

(73) Assignee: Galvanize Therapeutics, Inc., Redwood City, CA (US)

(21) Appl. No.: 18/533,642

(22) Filed: Dec. 8, 2023

Related U.S. Application Data
(63) Continuation of application No. PCT/US2022/032981, filed on Jun. 10, 2022.
(60) Provisional application No. 63/209,319, filed on Jun. 10, 2021.

(51) Int. Cl. A61B 18/12; A61B 18/00; A61B 18/14

(52) U.S. Cl. CPC A61B 18/1206; A61B 18/1492; A61B 2018/00214; A61B 2018/00345; A61B 2018/00702; A61B 2018/1472; A61B 2018/1475

(57) ABSTRACT

Devices, systems and methods are provided to treat damaged, diseased, abnormal, obstructive, undesired or potentially undesired tissue by delivering specialized pulsed electric field (PEF) energy and optionally therapeutic agents to target tissue areas, particularly within the vasculature. The therapy may be used to treat a variety of conditions, particularly conditions of the vascular system such as atherosclerosis and angina. Once a target tissue area has been identified as associated with vascular spasm, the target tissue areas are treated with PEF energy. Optionally, one or more agents can be delivered as well in conjunction with the treatment. Although the primary focus is on treating the coronary arteries, such treatment may be applicable to other portions of the vasculature, including the peripheral vasculature. Likewise, such treatment may be applicable to other body lumens.

### CROSS-REFERENCE TO RELATED APPLICATIONS

This application is a continuation of PCT Application No. PCT/US2022/032981, filed Jun. 10, 2022, which claims priority to and the benefit of U.S. Provisional Patent Application No. 63/209,319, filed Jun. 10, 2021, the content of which are fully incorporated herein by reference.

### BACKGROUND

Angina is a type of chest pain characterized by discomfort, pressure, squeezing, burning or fullness. In addition, pain may extend to the arms, neck, jaw shoulder or back. Likewise, other symptoms may be present such as dizziness, fatigue, nausea, shortness of breath and sweating. Angina is not a disease but an indication of an underlying heart problem.

There are multiple types of angina including stable angina (angina pectoris), unstable angina, variant angina and microvascular angina. Stable angina is the most common form of angina and is due to coronary artery disease. Coronary artery disease develops when the major blood vessels that supply the heart become damaged or diseased. This is typically caused by cholesterol-containing deposits known as plaque that builds up and narrows the arteries over time. This decreases the blood flow and eventually the reduced blood flow causes angina and other symptoms. A complete blockage can cause a heart attack.

Unstable angina is caused by blood clots that block an artery partially or completely. Blood clots may form, partially dissolve, and later form again and angina can occur each time a clot blocks blood flow in an artery. Thus, unstable angina typically causes unexpected chest pain. The blood clots are typically caused by rupture of narrowed coronary arteries causing injury to the coronary blood vessel resulting in blood clotting.

Variant angina is also known as Prinzmetal angina, Prinzmetal's variant angina and angina inversa. The pain from variant angina is caused by a spasm in the coronary arteries which supply blood to the heart muscle. The coronary arteries can spasm as a result of exposure to cold weather, stress, medications that tighten or narrow blood vessels, smoking and cocaine use.

Microvascular angina is typically a symptom of coronary microvascular disease (MVD). Coronary MVD (sometimes called small artery disease or small vessel disease) is heart disease that affects the walls and inner lining of tiny coronary artery blood vessels that branch off from the larger coronary arteries. In coronary MVD, the coronary artery blood vessels do not have plaque but have damage to the inner walls of the blood vessels that can lead to spasms and decreased blood flow to the heart muscle. In addition, abnormalities in smaller arteries that branch off of the main coronary arteries may also contribute to coronary MVD.

There are various conventional treatment options for angina. In stable angina, the uncomfortable symptoms are usually predictable and manageable. Normally this type of chest discomfort is relieved with rest, nitroglycerin or both. Nitroglycerin relaxes the coronary arteries and other blood vessels, reducing the amount of blood that returns to the heart and easing the workload of the heart. By relaxing the coronary arteries, it increases the blood supply to the heart.

Stable angina can develop into unstable angina which is typically noticed as feeling chest pain more easily and more often. Treatment for unstable angina is an emergency situation and involves locating the blood clot or closed artery and reestablishing blood flow therethrough. In some instances, percutaneous coronary intervention (PCI) may be required to open a blocked coronary artery. Briefly, this procedure involves undergoing cardiac catheterization for balloon angioplasty. Using a catheter with a small inflatable balloon at the tip, the balloon is inflated so as to compress the fatty plaque deposit located on the inner lining of the coronary artery. This procedure is often followed by insertion of a stent to then keep the coronary artery vessel propped open to allow for improved blood flow to the heart muscle. However, the increase in diameter can result in denudation of the endothelium, disruption of the internal elastic membrane and the media, and damage to about 20% of the smooth muscle cells (SMCs) in the media. Stents are designed to be expanded within a stenotic area in order to hold it open. However, stents also lead to a disruption of normal vasculature. The use of angioplasty to expand the vessel wall with the stent will have similar effects to those previously described, and self-expanding stents can continue expanding due to radial forces, prolonging disturbances to endothelial function. Furthermore, stents can disturb electrostatic equilibrium and prevent vascular spasm and elastic recoil, two important mechanical properties of arteries.

Changes to vessel architecture and cells from angioplasty and stenting can lead to the development of restenosis, a re-narrowing of the vessel at the site of intervention due to the iatrogenic injury response of the blood vessel. This problem also exists in the peripheral vasculature. Restenosis involves two major processes, arterial remodeling and neointimal hyperplasia. Arterial remodeling is a natural compensatory response, where the arteries enlarge in response to plaque formation to reduce vessel narrowing. However, in response to angioplasty, negative remodeling can lead to vessel constriction, reducing the overall vessel lumen. This is believed to be a primary mechanism for angioplasty restenosis, while in-stent restenosis appears to result primarily from neointimal hyperplasia. Damaged endothelial cells from angioplasty and stent insertion may further contribute to smooth muscle cell proliferation and migration by decreasing their nitric oxide production, a chemical known to inhibit smooth muscle cell growth.

In other instances, coronary artery bypass graft surgery may be utilized depending on the extent of coronary artery blockages and medical history. In this procedure, a blood vessel is used to route blood around the blocked part of the artery using grafting in an open surgical technique. Unfortunately, graft sources can cause donor site morbidity, have inadequate vessel architecture for grafting, or may be insufficient for multiple revascularization procedures. An additional open surgical technique is endarterectomy where the diseased blood vessel region is exposed and the plaque is physically removed. However, this technique is also highly invasive and carries risk of stroke, massive bleeding, and damage to the cranial nerves.

Since variant angina and microvascular angina are caused by spasms, these conditions are typically treated with medications to control the spasms. Drugs such as calcium antagonists and nitrates are the mainstays of treatment.

Typical antianginal drug therapies include β-adrenergic receptor blockers (beta-blockers), calcium channel blockers, and short-acting nitroglycerine. Beta-blockers can reduce the occurrence of angina episodes, improve ischaemic threshold, and even improve endothelial function in some patients through a possible antioxidant effect, although this remains to be proven widely in a clinical setting. The choice of drug class will be dependent upon patient tolerance and preference, contraindications, and the presence of comorbidities and are not recommended for patients with vasospastic angina. Also, abrupt withdrawal may result in rebound myocardial ischaemia.

Calcium channel blockers reduce afterload and increase the myocardial blood flow, while reducing heart rate and contractility. Systemic and coronary vasodilation is achieved through interaction with L-type Ca2+ receptors. It is preferable to use long-acting preparations and contraindications for nondihydropyridine calcium channel blockers are similar to those for beta-blockers.

Organic nitrates can reduce myocardial oxygen demands while maintaining or increasing coronary artery flow and are longstanding treatment for angina pectoris. Their safety profile allows them to be used with both beta-adrenergic or calcium-channel blockers, although several studies report no benefit in patients with chest pain and no obstructive coronary artery disease. Treatment with oral nitrates necessitates an individualized approach. Usually, angina symptoms will improve with oral nitrate therapy, especially in patients who benefit from sublingual nitrates. In some patients, angina may deteriorate, presumably due to a steal phenomenon by vasodilatation of collateral vessels. Some patients may not tolerate statins because of side effects. Statins, angiotensin-converting enzyme inhibitors (ACE-I), and low-dose aspirin are also current pharmacology for coronary microvascular dysfunction with the aim of treating microvascular endothelial dysfunction. Patients with exercise-induced ischemia and flow-mediated dilation respond to statin therapy, and the observed beneficial effects are considered to be attributable to improved endothelial function. ACE-I medication is effective in patients with MVA with improvement to coronary flow reserve in this patient population. If patients are intolerant of ACE-I, angiotensin receptor blockers are an alternative, although there are no data for how effective this therapy may be for coronary microvascular dysfunction patient subgroups. Additionally, for patients with insulin resistance, metformin administration is effective in increasing microvascular function. Imipramine, a tricyclic medication, also improved the symptoms of patients with chest pain and no obstructive coronary artery disease; this was thought to be due to a visceral analgesic effect. Aspirin may be beneficial in patients with coronary artery disease but when coronary artery disease is excluded there is no evidence to support treatment with aspirin. In this case, the risk of bleeding events outweighs any theoretical benefits. For individuals with MVA there is no conclusive evidence that supports a specific class of drugs or combined therapy, or therapy and technology, presumably because of the knowledge gap regarding the cause of MVA and the inconsistency of patient response to available drug treatments.1

Most types of angina are chronic conditions, either themselves or based on underlying chronic diseases. Likewise, atherosclerosis is typically chronic and progressive. Although various treatments are available for each of these conditions, either separately or combined, such treatments include risks, adverse effects and are typically not curative, involving ongoing treatment. Therefore, improvements in in the treatment of these conditions and vascular conditions are desired. Such treatments should be safe, effective, and lead to reduced complications. At least some of these objectives will be met by the systems, devices and methods described herein.

### SUMMARY OF THE INVENTION

Described herein are embodiments of apparatuses, systems and methods for treating target tissue. Likewise, the invention relates to the following numbered clauses:

In a first aspect, a system is provided for treating a blood vessel comprising an energy delivery catheter comprising an energy delivery body positionable within or near the blood vessel, and a generator in electrical communication with the energy delivery body, wherein the generator includes at least one energy delivery algorithm that provides pulsed electric field energy to the energy delivery body so that the energy treats cells of the blood vessel while maintaining an extracellular matrix of the blood vessel in a manner that decreases or eliminates the ability of the blood vessel to vasconstrict. It may be appreciated that near is considered to be at a distance in which the target tissue, such as the blood vessel or targeted portion of the blood vessel, is able to receive energy delivered from the energy delivery body in a manner so as to achieve the desired outcome.

In some embodiments, treating comprises removing, destroying or killing the cells. In some embodiments, the cells comprise vascular smooth muscle cells involved in vasoconstriction of the blood vessel. In some embodiments, treating comprises disrupting local innervation to the vascular smooth muscle cells. Optionally, disrupting local innervation comprises disrupting a neural pathway from a local area of spasm through at least one vagal afferent fiber that contributes to anginal pain symptoms. In other embodiments, the cells comprise endothelial cells and maintaining the extracellular layer leads to regeneration of an endothelial layer of the blood vessel.

In some embodiments, the energy delivery catheter is configured to deliver a fluid. Optionally, the fluid comprises a conductive solution. In some instances, the energy delivery catheter is configured to deliver the energy and the fluid in a manner so that the fluid acts as a virtual electrode. In some instances, the algorithm is configured to trigger delivery of the energy and the fluid in a timing sequence so that the fluid acts as a virtual electrode.

In some embodiments, the fluid comprises an agent. In some embodiments, the agent comprises a chemical, drug, medication, chemotherapy agent, immunotherapy agent, micelle, liposome, embolic, nanoparticle, drug-eluting particle, gene, plasmid, protein or combination of these. In some embodiments, treating comprises causing the cells to uptake the agent. In some embodiments, uptake of the agent inhibits hyperplastic regrowth of the cells.

In some embodiments, the energy delivery body comprises an expandable member surrounding at least one electrode. In some embodiments, the energy delivery body comprises a shaft, wherein the at least electrode comprises a plurality of electrodes disposed along the shaft, and wherein the expandable member comprises an elongate expandable member surrounding the plurality of electrodes.

In some embodiments, the expandable member is configured to weep fluid. In some embodiments, the energy delivery body includes at least one port within the expandable member through which fluid is deliverable so as to weep from the expandable member. In some embodiments, the energy delivery body comprises an electrode formed by a plurality of ribbons or wires. In some embodiments, the energy delivery body comprises one or more protrusions, wherein each protrusion bends radially outward from a longitudinal axis of the energy delivery catheter. In some embodiments, the one or more protrusions comprise one or more prongs.

In some embodiments, the pulsed electric field energy is generated from a waveform including one or more of the following parameters a) a voltage in a range of 500-3000V, b) a frequency in a range of 100-800 kHz, c) a total on-time in a range of 25-250 μs, and d) packets per electrode activation are in a range of 1-30 packets.

In some embodiments, the voltage is in a range of 2000-2500V. In some embodiments, the frequency is in a range of 400-600 kHz. In some embodiments, the total on-time is in a range of 50-150 μs. In some embodiments, the packets per electrode activation are in a range of 1-10 packets. In some embodiments, the pulsed electric field energy is generated from a waveform including one or more of the following parameters: a) a voltage in a range of 1000-6000V, b) a frequency in a range of 10-500 kHz, c) a total on-time in a range of 50-500 μs, and d) packets per electrode activation are in a range of 1-30 packets. In some embodiments, the pulsed electric field energy causes disruption of calcification. In some embodiments, the voltage is in a range of 2000-4000V. In some embodiments, the frequency is in a range of 50-250 KHz. In some embodiments, the total on-time is in a range of 75-150 μs.

In a second aspect, a system is provided for treating a vascular spasm comprising an energy delivery catheter comprising an energy delivery body positionable on or near muscular tissue, such as cardiac myocardium, and a generator in electrical communication with the energy delivery body, wherein the generator includes at least one energy delivery algorithm that provides pulsed electric field energy to the energy delivery body so that the energy modifies the muscular tissue so as to replace or eliminate regional mechanical forces that cause the vascular spasm. Again, it may be appreciated that near is considered to be at a distance in which the target tissue, such as the cardiac myocardium or targeted portion of the cardiac myocardium, is able to receive energy delivered from the energy delivery body in a manner so as to achieve the desired outcome.

In some embodiments, the energy delivery catheter is configured to deliver a fluid. In some embodiments, the fluid comprises a conductive solution. In some embodiments, the energy delivery catheter is configured to deliver the energy and the fluid in a manner so that the fluid acts as a virtual electrode. In some embodiments, the algorithm is configured to trigger delivery of the energy and the fluid in a timing sequence so that the fluid acts as a virtual electrode.

In some embodiments, the fluid comprises an agent. In some embodiments, the agent comprises a chemical, drug, medication, chemotherapy agent, immunotherapy agent, micelle, liposome, embolic, nanoparticle, drug-eluting particle, gene, plasmid, protein or combination of these. In some embodiments, treating comprises causing the cells to uptake the agent. In some embodiments, the pulsed electric field energy is generated from a waveform including one or more of the following parameters: a) a voltage in a range of 500-3000V, b) a frequency in a range of 100-800 kHz, c) a total on-time in a range of 25-250 μs, and d) packets per electrode activation are in a range of 1-30 packets.

In some embodiments, the voltage is in a range of 2000-2500V. In some embodiments, the frequency is in a range of 400-600 kHz. In some embodiments, the total on-time is in a range of 50-150 μs. In some embodiments, the packets per electrode activation are in a range of 1-10 packets.

In a third aspect of the present invention, a system is provided for treating anginal pain symptoms comprising an energy delivery catheter comprising an energy delivery body positionable within or near the blood vessel; and a generator in electrical communication with the energy delivery body, wherein the generator includes at least one energy delivery algorithm that provides pulsed electric field energy to the energy delivery body so that the pulsed electric field energy disrupts a neural pathway through at least one vagal afferent fiber that contributes to the anginal pain symptoms. In some embodiments, the neural pathway innervates vascular smooth muscle cells of a blood vessel associated with the anginal pain symptoms.

In a fourth aspect of the present invention, a system is provided for treating a portion of a blood vessel comprising an electrode positionable near the portion of the blood vessel; and a generator in electrical communication with the electrode, wherein the generator includes at least one energy delivery algorithm that provides pulsed electric field energy to the electrode so that the energy treats the portion of the blood vessel. In some embodiments, the pulsed electric field energy is generated from a waveform including one or more of the following parameters: a) a voltage in a range of 500-3000V, b) a frequency in a range of 100-800 kHz, c) a total on-time in a range of 25-250 μs, and d) packets per electrode activation are in a range of 1-30 packets.

In some embodiments, the pulsed electric field energy is generated from a waveform including one or more of the following parameters: a) a voltage in a range of 1000-6000V, b) a frequency in a range of 10-500 kHz, c) a total on-time in a range of 50-500 μs, and d) packets per electrode activation are in a range of 1-30 packets. In some embodiments, the energy delivery body comprises an expandable member surrounding at least one electrode. In some embodiments, the energy delivery body comprises a shaft, wherein the at least electrode comprises a plurality of electrodes disposed along the shaft, and wherein the expandable member comprises an elongate expandable member surrounding the plurality of electrodes.

In a sixth aspect of the present invention, a method is provided for treating a blood vessel comprising positioning an energy delivery body of an energy delivery catheter within or near the blood vessel, and delivering pulsed electric field energy to the energy delivery body so that the energy treats cells of the blood vessel while maintaining an extracellular matrix of the blood vessel in a manner that decreases or eliminates the ability of the blood vessel to vasconstrict.

In a seventh aspect of the present invention, a method is provided for treating a vascular spasm comprising positioning an energy delivery body of an energy delivery catheter on or near cardiac myocardium and delivering pulsed electric field energy to the energy delivery body so that the energy modifies the cardiac myocardium so as to replace or eliminate regional mechanical forces that cause the vascular spasm.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 provides an overview illustration of an example therapeutic system for use in delivering the specialized PEF energy to the vasculature.

FIG. 2 illustrates an embodiment of an energy delivery catheter having an energy delivery body comprised of an elongate expandable member surrounding a plurality of electrodes.

FIG. 3 illustrates an embodiment of an energy delivery catheter having an energy delivery body comprising an electrode formed by a plurality of ribbons or wires.

FIGS. 4-5 illustrates embodiments of energy delivery bodies comprising one or more protrusions wherein each protrusion bends radially outward from the longitudinal axis or shaft of the catheter.

FIGS. 6A-6B illustrates an embodiment of an energy delivery body comprising one or more prongs which act as protrusions.

FIG. 7 illustrates another embodiment of a catheter comprising an energy delivery body having a shape configured for endovascular treatment.

FIGS. 8-12 illustrate example embodiments of catheters that deliver agents through or near the energy delivery body.

FIG. 13 includes a schematic illustration of a wall of a coronary artery and an embodiment of an energy delivery body positioned adjacent the wall.

FIG. 14 illustrates an embodiment of a waveform of a signal prescribed by an energy delivery algorithm.

FIG. 15 illustrates various examples of biphasic pulses (comprising a positive peak and a negative peak) having a switch time therebetween.

### DETAILED DESCRIPTION

Devices, systems and methods are provided to treat damaged, diseased, abnormal, obstructive, undesired or potentially undesired tissue by delivering specialized pulsed electric field (PEF) energy and optionally therapeutic agents to target tissue areas, particularly within the vasculature. Although such devices, systems and methods will be primarily focused on treating the coronary arteries, such treatment may be applicable to other portions of the vasculature, including the peripheral vasculature. Likewise, such treatment may be applicable to other body lumens.

The PEF energy is delivered to the target tissue areas in a manner so as to create a non-thermal treatment (i.e. energy is below a threshold for causing thermal ablation or below a threshold for causing extracellular protein denaturation implicated in clinical morbidity of thermal therapy outcomes). Consequently, when extracellular matrices are present, the extracellular matrices are preserved, and the targeted tissue maintains its structural architecture. Thus, portions of the blood vessel walls are able to be preserved which are critical to maintaining the integrity and functionality of the tissue. This provides a number of benefits. To begin, this allows for the treatment of tissues that are often considered untreatable by conventional methods. Target tissues that are near sensitive structures are typically considered untreatable due to proximity to the sensitive structures. Likewise, many conventional therapies are contraindicated due to the potential for damage to the sensitive structures by the therapy or because the therapies are deemed ineffective due to the proximity of the sensitive structures. Once tissue is treated, the survival of the structural architecture also allows for the natural influx of biological elements, such as components of the immune system, or for the introduction of various agents to further the therapeutic treatment. This provides a number of treatment benefits as will be described in more detail in later sections.

### Therapeutic System and Devices

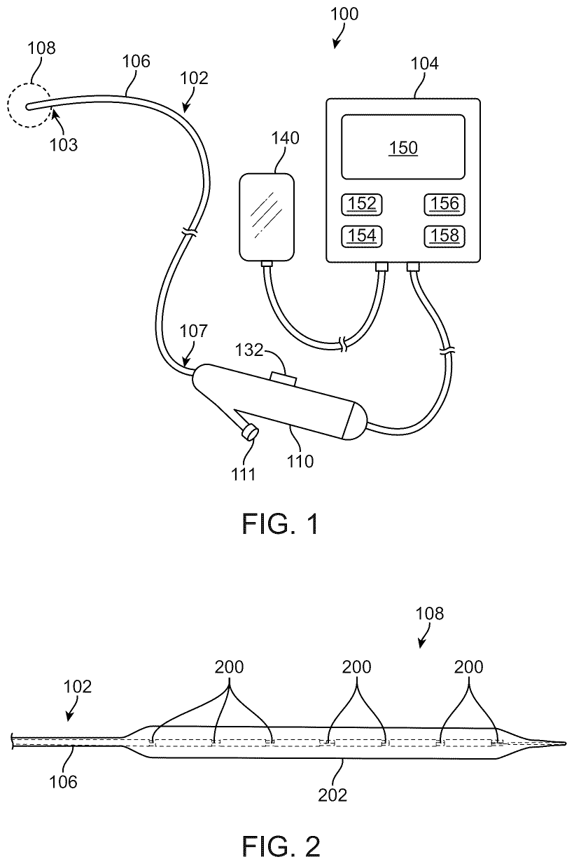

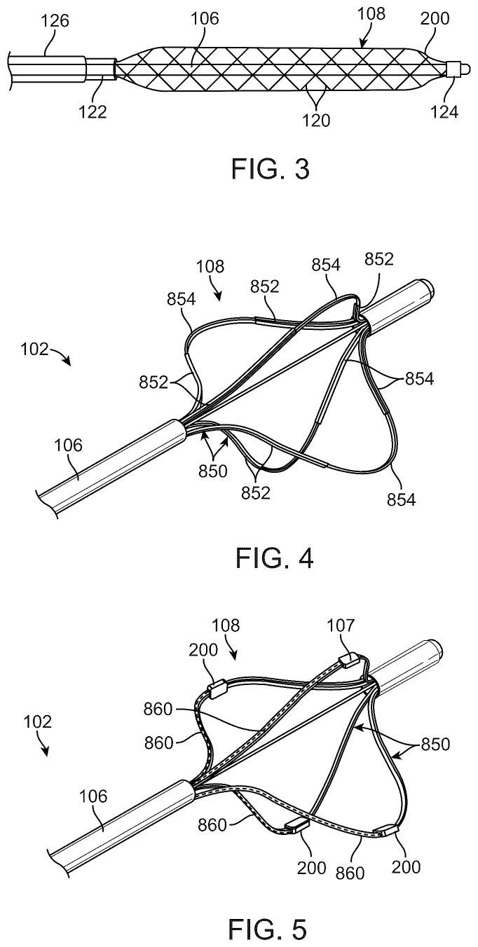

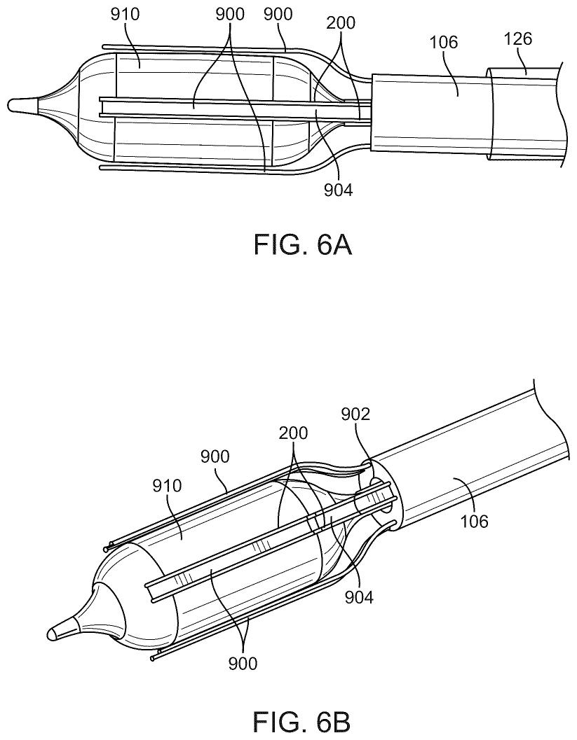

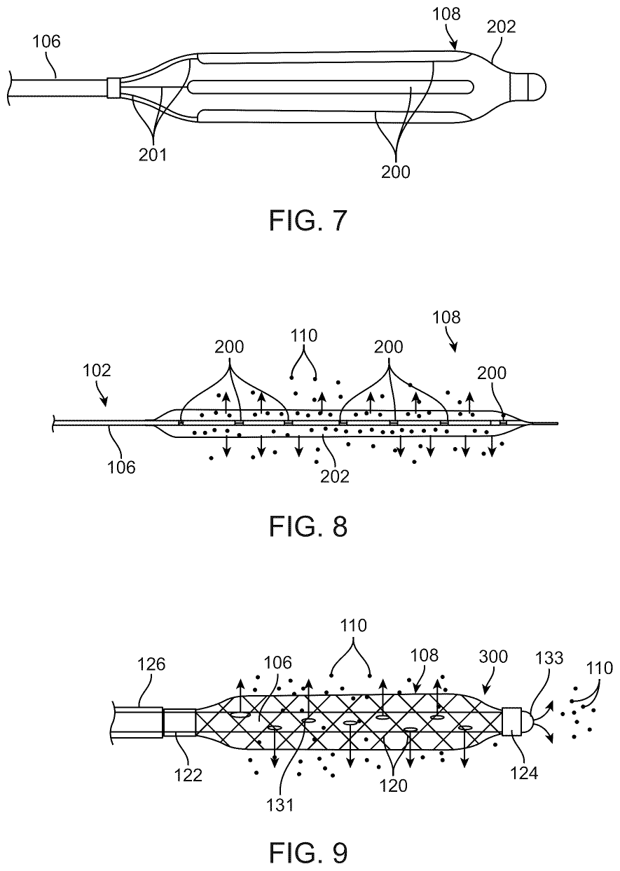

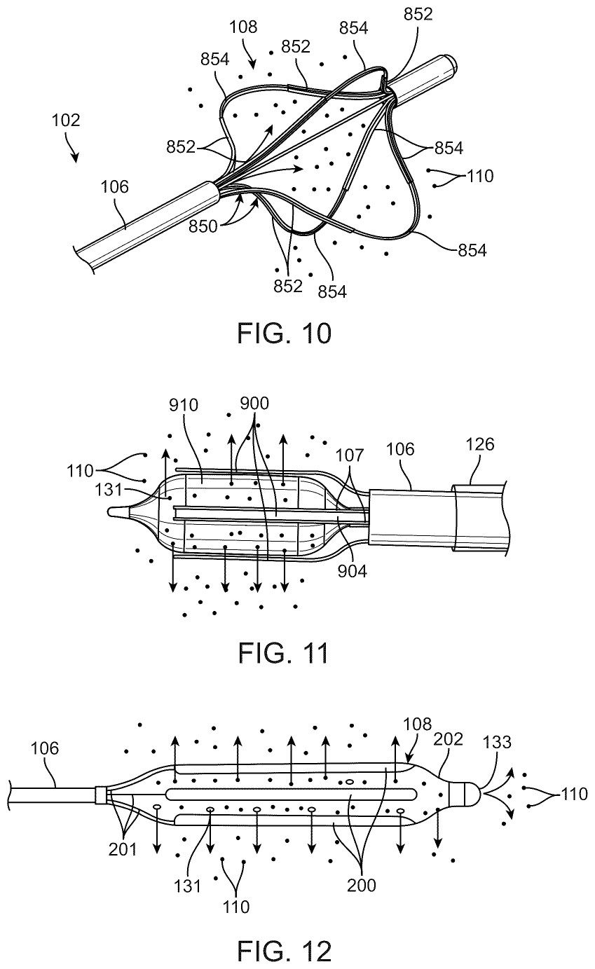

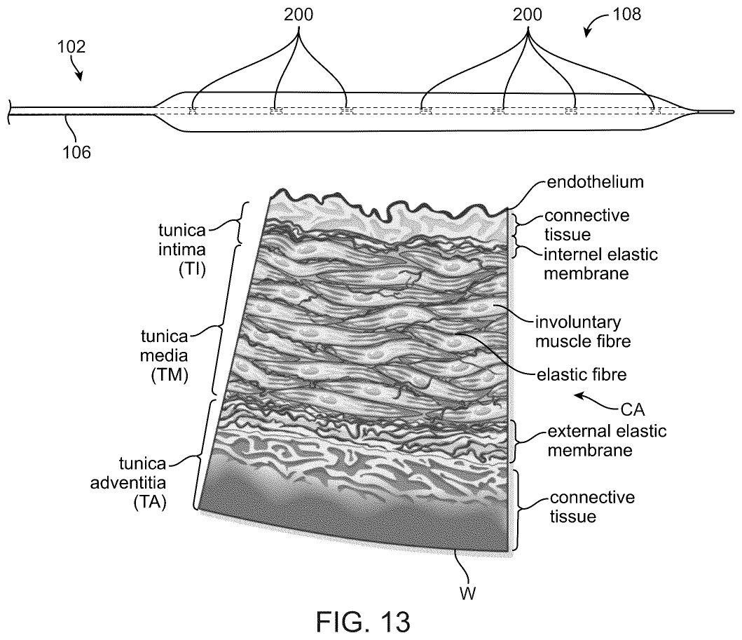

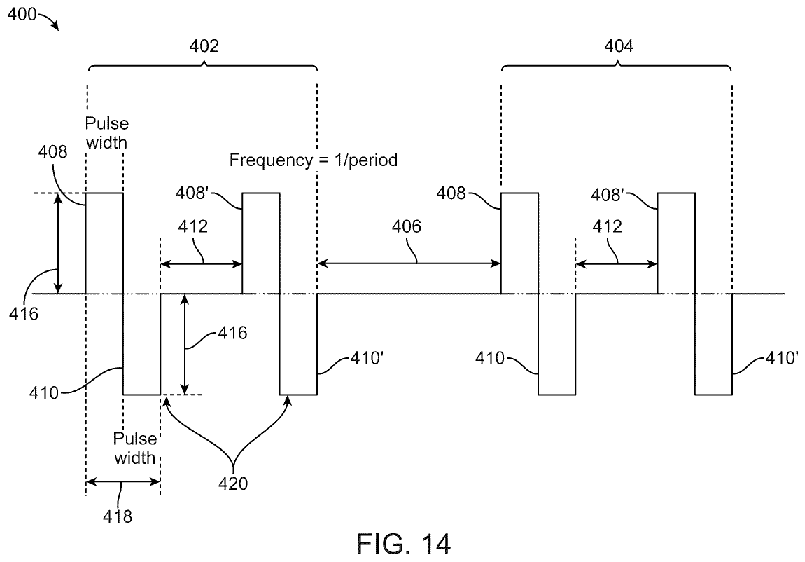

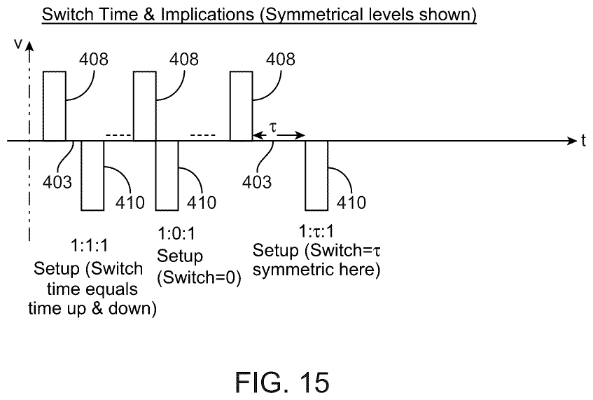

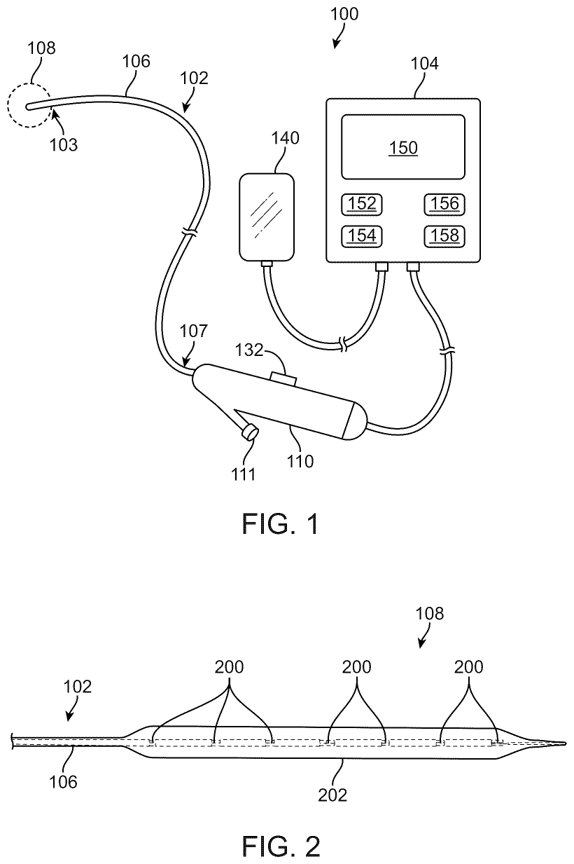

FIG. 1 provides an overview illustration of an example therapeutic system 100 for use in delivering the specialized PEF energy to the vasculature. In this embodiment, the system 100 comprises an energy delivery catheter 102 comprising a shaft 106 having a distal end 103 and a proximal end 107. The catheter 102 includes an energy delivery body 108 which is generically illustrated as a dashed circle near the distal end 103 of the shaft 106. It may be appreciated that the energy delivery body 108 may take a variety of forms having structural differences which encumber the drawing of a single representation, however individual example embodiments will be described and illustrated herein. The energy delivery body 108 may be mounted on or integral with an exterior of the shaft 106 so as to be externally visible. Or, the energy delivery body 108 may be housed internally within the shaft 106 and exposed by advancing from the shaft 106 or retracting the shaft 106 itself. Likewise, more than one energy delivery body 108 may be present and may be external, internal or both. In some embodiments, the shaft 106 is comprised of a polymer, such as an extruded polymer. It may be appreciated that in some embodiments, the shaft 106 is comprised of multiple layers of material with different durometers to control flexibility and/or stiffness. In some embodiments, the shaft 106 is reinforced with various elements such as individual wires or wire braiding. In either case, such wires may be flat wires or round wires. Wire braiding has a braid pattern and in some embodiments the braid pattern is tailored for desired flexibility and/or stiffness. In other embodiments, the wire braiding that reinforces the shaft 106 may be combined advantageously with multiple layers of material with different durometers to provide additional control of flexibility and/or stiffness along the length of the shaft.

In some embodiments, the catheter 102 is delivered through a delivery device during a suitable access procedure. When accessing the coronary arteries, the heart is typically accessed via the femoral artery or radial artery by an access procedure, such as the Seldinger technique. A sheath is inserted into the artery which acts as a conduit through which various catheters and/or tools may be advanced, including the treatment catheter 102 and optionally a device for delivering an agent. It may be appreciated that in some embodiments, the treatment catheter 102 and agent delivery are combined into a single device. Typically, the coronary vasculature is approached with the use of X-ray imaging. Once the desired treatment locations are identified, the treatment catheter 102 is utilized to deliver the treatment energy.

Each energy delivery body 108 comprises at least one electrode for delivery of the PEF energy. Typically, the energy delivery body 108 comprises a single delivery electrode and operates in a monopolar arrangement which is achieved by supplying energy between the energy delivery body 108 disposed near the distal end 103 of the catheter 102 and a return electrode 140 positioned upon the skin of the patient. In some embodiments, the energy delivery body 108 provides 32 mm² exposed surface area for energy delivery. It will be appreciated, however, that bipolar energy delivery and other arrangements may alternatively be used. When using bipolar energy delivery, the catheter 102 may include a plurality of energy delivery bodies 108 configured to function in a bipolar manner or may include a single energy delivery body 108 having multiple electrodes configured to function in a bipolar manner. Likewise, the plurality of energy delivery bodies 108 may be on separate instruments. The catheter 102 typically includes a handle 110 disposed near the proximal end 107. The handle 110 is used to maneuver the catheter 102 and may include an actuator 132 for manipulating the energy delivery body 108. In some embodiments, the energy delivery body 108 transitions from a closed or retracted position (during access) to an open or exposed position (for energy delivery) which is controlled by the actuator 132. Thus, the actuator 132 typically has the form of a knob, button, lever, slide or other mechanism. It may be appreciated that in some embodiments, the handle 110 includes a port 111 for introduction of fluids, agents, substances, tools or other devices for delivery through the catheter 102. Example fluids include suspensions, mixtures, chemicals, liquids, agents, chemotherapy agents, immunotherapy agents, micelles, liposomes, embolics, nanoparticles, drug-eluting particles, genes, plasmids, and proteins, to name a few.

As an added measure it may be desirable to control any potential degree of temperature rise during treatment delivery. In some embodiments, this is achieved with the use of internally cooled or irrigated (open-system) electrodes. Further, the cooling irrigation may optionally contain secondary biologic compounds utilized by the PEF therapy, such as genetic material, chemotherapy, or immunostimulants. The electrodes may be pre-cooled, cooled during delivery, or cooled afterwards. The coolant may be chilled, room temperature, normothermic temperature, or at deliberately elevated temperature. In some embodiments, the irrigation material also includes calcium or other materials to increase treatment efficacy. In some embodiments, the irrigation material uses a hypertonic solution to regionally increase the electrical conductivity, serving as a virtual electrode to extend the applicable treatment zone.

The catheter 102 is in electrical communication with a generator 104 which is configured to generate the PEF energy. In this embodiment, the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, the user interface 150 on the generator 104 is used to select the desired treatment algorithm 152. In other embodiments, the algorithm 152 is automatically selected by the generator 104 based upon information obtained by one or more sensors, which will be described in more detail in later sections. A variety of energy delivery algorithms may be used. In some embodiments, one or more capacitors are used for energy storage/delivery, however any other suitable energy storage element may be used. In addition, one or more communication ports are typically included.

In this embodiment, the generator 104 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included.

In some embodiments, the generator 104 includes three sub-systems: 1) a high-energy storage system. 2) a high-voltage, medium-frequency switching amplifier, and 3) the system controller, firmware, and user interface. In some embodiments, the system controller includes a cardiac synchronization trigger monitor that allows for synchronizing the pulsed energy output to the patient's cardiac rhythm. In other embodiments, energy delivery is triggered by other monitoring mechanisms or simply by direct input from the operator. The generator takes in alternating current (AC) mains to power multiple direct current (DC) power supplies. Alternately, the generator may take in DC power from a battery or other electrical delivery system. The generator's controller can cause the DC power supplies or battery to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only, and systems delivering pulsed electric fields may or may not include additional switching amplifier components.

The user interface 150 can include a touch screen and/or more traditional buttons or a mouse to allow for the operator to enter patient data, select a treatment algorithm (e.g., energy delivery algorithm 152), initiate energy delivery, view records stored on the storage/retrieval unit 156, and/or otherwise communicate with the generator 104. The user interface 150 can include a voice-activated mechanism to enter patient data or may be able to communicate with additional equipment in the suite so that control of the generator 104 is through a secondary separate user interface.

In some embodiments, the user interface 150 is configured to receive operator-defined inputs. The operator-defined inputs can include a duration of energy delivery, one or more other timing aspects of the energy delivery pulse, power, and/or mode of operation, or a combination thereof. Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination or series of subcombinations thereof.

In some embodiments, the system 100 also includes a mechanism for acquiring an electrocardiogram (ECG), such as an external cardiac monitor 170. Example cardiac monitors are available from AccuSync Medical Research Corporation. In some embodiments, the external cardiac monitor 170 is operatively connected to the generator 104. The cardiac monitor 170 can be used to continuously acquire an ECG signal. External electrodes 172 may be applied to the patient P to acquire the ECG. The generator 104 analyzes one or more cardiac cycles and identifies the beginning of a time period during which it is safe to apply energy to the patient P, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. It will be appreciated that such cardiac synchronization is typically utilized when using monopolar energy delivery, however it may be utilized as part of other energy delivery methods.

FIG. 2 illustrates an embodiment of an energy delivery catheter 102 having an energy delivery body 108 comprised of an elongate expandable member 202 surrounding one or more electrodes 200. In this embodiment, a plurality of electrodes 200 are disposed along the shaft 106 near its distal end. In this embodiment, the plurality of electrodes 200 are equally spaced, however it may be appreciated that the electrodes 200 may be disposed with regard to any desired pattern of spacing therebetween. The electrodes 200 are surrounded by an expandable member 202, such as a balloon, wherein the expandable member 202 is expandable, such as to contact the walls of a blood vessel when inserted therein. In this embodiment, the expandable member 202 is inflatable with a conductive solution. Thus, when the expandable member 202 expands to contact the walls of the blood vessels, energy delivered through the one or more electrodes 200 is transmittable through the conduction solution to the walls of the blood vessels. It may be appreciated that conduction to the walls of the blood vessels may also be achieved without contact or with minimal contact, such as when blood within the blood vessels acts as a conductive path to the walls of the blood vessel.

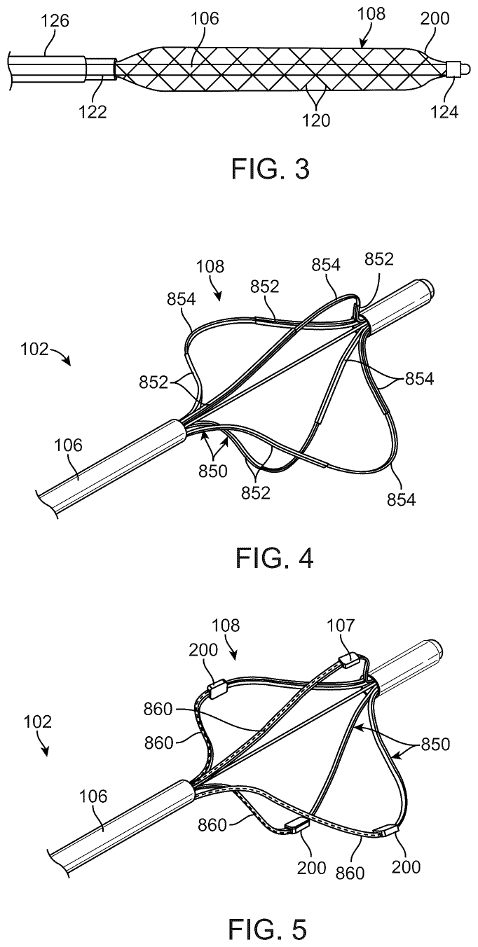

FIG. 3 illustrates an embodiment of an energy delivery catheter 102 having an energy delivery body 108 comprising an electrode 200 formed by a plurality of ribbons or wires 120, wherein the energy delivery body 108 is mounted on a shaft 106 which extends through the energy delivery body 108. In this embodiment, the energy delivery body 108 has a basket shape constrained by a proximal end constraint 122 and a distal end constraint 124. In this configuration, in order for the energy delivery body 108 to collapse, either the proximal end constraint 122 or distal end constraint 124 slides freely on the shaft 106 while the other end is fixedly attached to the shaft 106. Upon positioning of the energy delivery body 108 in the target treatment area, a sheath 126 is withdrawn by the operator via, for example, a lever or slider or plunger of the catheter's handle 110, which is operatively connected to the sheath 126. The withdrawal of the sheath 126 removes the restraint keeping the energy delivery body 108 collapsed, thus allowing its expansion leading to the wires 120 of the energy delivery body 108 contacting the blood vessel walls.

In some embodiments, the collapsed configuration of the energy delivery body 108 can be achieved by mechanisms which restrict its expansion without the use of a sheath 126. For example, in some embodiments, a pull wire is attached to the proximal end constraint 122 of the energy delivery body 108 and extends down a lumen along the shaft 126 where it is operatively connected to a lever, slider, or plunger of the catheter's handle 110. In this embodiment, the distal end constraint 124 is fixedly attached to the shaft 106 and the proximal end constraint 122 is configured to slide freely on the shaft 106. While the pull wire is under pull force, the proximal end constraint 122 is positioned so that the energy delivery body 108 is collapsed. The pull wire can be maintained in this position by restraint within the handle 110. Release of the pull force, such as by reduction or removal of the restraint within the handle 110, allows the pull wire to move, thus freeing the proximal end constraint 122 and allowing it to travel closer to its distal end constraint 124 as self-expanding properties of the energy delivery body 108 cause expansion.

In other embodiments, the proximal end constraint 122 is affixed to the shaft 106 and the distal end constraint 124 is free to slide on the shaft 106. Further, a push rod (or tubing to achieve higher column strength) is attached to the distal end constraint 124 and extends down a lumen along the inner shaft 106 where it is operatively connected to mechanism such as a lever, slider, or plunger of the catheter's handle 110. When the push rod is pushed and subsequently restrained within the handle 110 of the catheter 102, the distal constraint 124 is moved away from the proximal end constraint 122 which causes the energy delivery body 108 to collapse. When the energy delivery body 108 is self-expanding, release of the push rod allows the energy delivery body 108 to expand. Alternatively, the push rod may be retracted, pulling the distal end constraint 124 toward the proximal end constraint 122 which causes the energy delivery body 108 to expand.

In some embodiments, the energy delivery body 108 is formed from a braided metal tube constrained at both the proximal end constraint 122 and the distal end constraint 124 and configured to form a basket. The energy delivery body 108 can be controlled (i.e., collapsed, deployed) as described above. When the energy delivery body 108 comprises a braided metal tube, each wire in the braided tube is supported by multiple wires next to it as well as by the interwoven nature of the braid itself. This support and interwoven configuration can assure minimal variation in space between wires, otherwise known as pore or opening size of the braid. In addition, this support and interwoven configuration can allow constructing the braided tube from very small wires and yet have significant radial stability of the basket. This allows the use of many wires (e.g., 12, 16, 18, 20, 22, 24, etc.) while maintaining a relatively small profile of the energy delivery body 108 in the collapsed/constrained state and optimizing the opening size of the braided tube when electrode(s) is/are deployed/expanded. In this embodiment, the space between wires is rather small, leading to a treatment that is essentially continuous over 360 degrees of the inner lumen of a blood vessel.

FIG. 4 illustrates another embodiment of an energy delivery body 108. In this embodiment, the energy delivery body 108 comprises one or more protrusions 850 rather than a basket weave. Each protrusion 850 is formed by a wire or ribbon 120 which acts as an electrode and bends radially outward from the longitudinal axis or shaft 106 of the catheter 102. In this embodiment, each protrusion 850 is electrically isolated from each of the other protrusions. The protrusions 850 may be comprised of a variety of suitable materials so as to act as an electrode, such as stainless steel, spring steel, or other alloys, and may be, for example, round wires or ribbon. Each protrusion 850 is insulated with a segment of insulation 852, such as a polymer (e.g., PET, polyether block amide, polyimide), over at least a portion of the proximal and distal ends of the energy delivery body 108. The exposed portion 854 of the wire or ribbon can then act as an electrode on each protrusion 850. In one embodiment, the exposed portions 854 of the protrusions 850 are completely free of insulation 852. In another embodiment, the insulation 852 is removed only from the outer surface of the protrusion 850 leaving the side of the protrusion 850 that does not come in contact with the tissue (e.g., an inner surface that faces the shaft 106 of the catheter 102) completely insulated. In one embodiment, each protrusion 850 is energized independently, with two protrusions 850 acting as neutral electrodes (return) and two protrusions 850 acting as active electrodes. Neutral and active electrodes can be positioned right next to each other. Neutral electrodes located 180 degrees from each other (opposite electrodes) can be electrically connected to each other and so can be the active electrodes. In this embodiment, only two conductive wires (power lines) are needed to connect two pairs of protrusions 850 to the generator 104. Further, pairs of protrusions 850 that are utilized in a bipolar fashion can further be multiplexed to allow for any combination or rotation of active versus neutral electrode. The generator 104 can be configured to have sufficient channels to support any of these approaches (i.e., 1 to 4 channels). This embodiment of the energy delivery body 108 can optionally be delivered in a collapsed configuration and expanded into tissue contact via a pullback wire and mechanism within the handle.

FIG. 5 illustrates another embodiment of energy delivery body 108 comprising one or more protrusions 850 wherein each protrusion 850 bends radially outward from the longitudinal axis or shaft 106 of the catheter 102. However, in this embodiment, each protrusion 850 is formed from a non-conductive material and carries, supports, and/or is otherwise coupled to a separate electrode 200. Each electrode 200 has a conductive wire 860 connecting the electrode 200 to the generator 104. The protrusions 850 position said electrodes 200 against the tissue upon expansion, such as via a pull wire and mechanism within the handle. In this embodiment, each electrode 200 is placed over or adjacent each protrusion 850. If the protrusions 850 are comprised of a metal, insulation is provided to electrically isolate the electrodes 200 from the protrusions 850 themselves. If the protrusions 850 are comprised of a polymer or other non-conductive material, additional insulation would not be required. In some embodiments, the protrusions 850 are comprised of round wire or ribbon and configured to form a straight basket, as shown. In other embodiments (not shown), the protrusions 850 are configured in a spiral shape. It may be appreciated that separate electrodes 200 as depicted in FIG. 5 may likewise be applied to other embodiments, such as wherein the basket is comprised of a braided material. Similar to the embodiment of FIG. 4, each electrode 200 may be energized in a variety of combinations. Furthermore, each protrusion 850) can carry the electrodes 200 that can be electrically connected to each other or electrically insulated from each other. To increase the surface area of the electrodes 200 each can be constructed from, for example, a metallic coil or in a form of a slotted (e.g. laser cut) tube. These configurations would allow for greater spatial coverage and yet maintain the flexibility of the electrodes 200 to allow the protrusions 850 of the basket to bend and straighten freely. As in FIG. 4, the surface of the protrusions 850 can be completely exposed or insulated over areas that do not come in contact with the tissue.

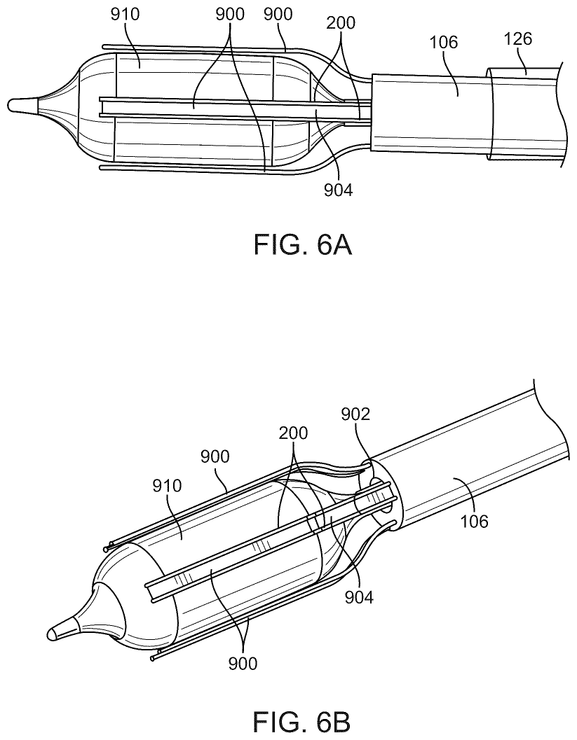

It may be appreciated that the energy delivery body 108 can be optimized for situations in which force exerted onto the vessel wall is desired to be more highly controlled. In this embodiment, the energy delivery body 108 is delivered into the vessel lumen via a three-step process. First, referring to FIGS. 6A-6B, a sheath 126 is withdrawn proximally thus exposing one or more prongs 900 which act as protrusions. This embodiment includes four prongs 900 arranged symmetrically around a central lumen 902. It may be appreciated that any number of prongs 900 may be present including one, two, three, four, five, six or more. Each prong 900 includes at least one electrode 200. FIG. 6A illustrates an embodiment of a prong 900 having two electrodes 200 having an elongate shape (such as wire) attached to an insulating substrate 904, such as a polymer substrate (e.g. ribbon, strip), therebetween as a means to maintain distance between the electrodes 200. It may be appreciated that the electrodes 200 may have a round or square/rectangular cross-section, and are typically affixed to the insulating substrate 904 such that the electrodes 200 are substantially parallel to one another. The manufacturing method of attaching the electrodes 200 to the insulating substrate 904 can employ (but is not limited to) co-extrusion, deposition, adhesive based bonding, and thermal bonding. The width of the insulting substrate 904 can vary. Electrodes 200 can be electrically connected to each other, can be insulated from each other or different patterns of electrical interconnection between electrodes depending on the energy application algorithm controlled by the generator.

Once the one or more prongs 900 are exposed, the second step of the three-step process involves introducing a separate expandable member 910, such as a balloon, by advancing the expandable member 910 from the lumen 902 while in an unexpanded state. The third step involves expanding the expandable member 910, such as inflating the balloon, as illustrated in FIGS. 6A-6B, until a desired interface between the prongs 900 (and therefore electrodes 200) and vessel wall is achieved. In another embodiment, the prongs 900 are positioned while the expandable member 910 is already disposed beneath the prongs 900 so their relative longitudinal position does not change. In this configuration, the withdrawal of the sheath 126 exposes both the expandable member 910 and the prongs 900 at the same time, thus eliminating the step of advancing the expandable member 910 out of the lumen 902. As described above, the expandable member 910 is subsequently expanded (e.g. inflated) until the desired interface between the prongs 900 and vessel wall is achieved. The size (e.g. length, width) of the prongs 900 can be the same or different. The number of prongs 900 can vary between 1 (monopolar configuration) and 100 (monopolar and/or bipolar) configuration. Energy application to the electrodes 200 can vary widely depending on the algorithm of the energy delivery apparatus (e.g. generator).

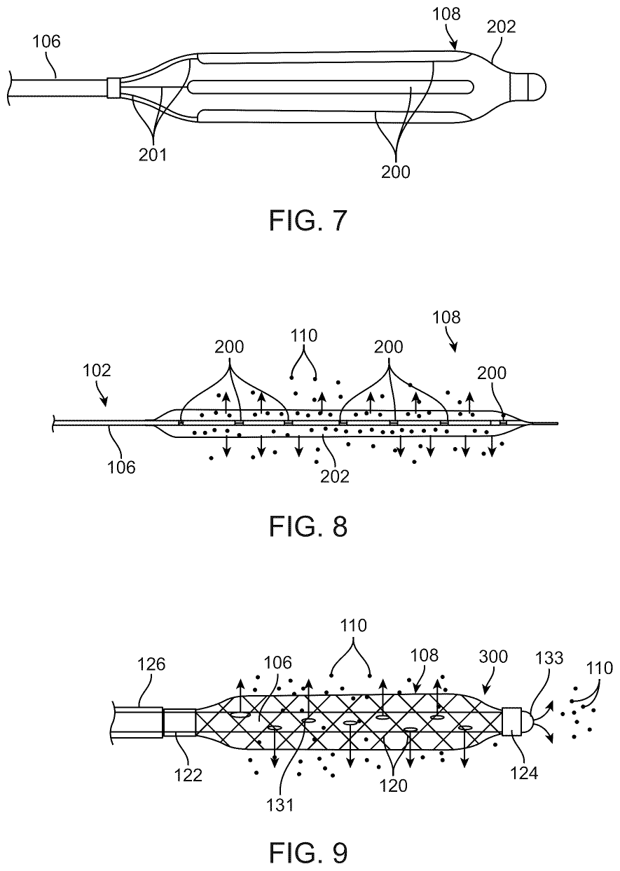

FIG. 7 illustrates another embodiment of a catheter 102 comprising an energy delivery body 108 having a shape configured for endovascular treatment. In this embodiment, the energy delivery body 108 comprises an expandable member 202, such as an inflatable balloon, having at least one electrode 200 mounted thereon or incorporated therein. The energy delivery body 108 is delivered to a targeted area in a collapsed configuration. In this embodiment, the electrode 200 has the form of a pad having a relatively broad surface area and thin cross-section. The pad shape provides a broader surface area than other shapes, such as a wire shape. Each electrode 200 is connected with a conduction wire 201 which electrically connects the electrode 200 with the generator. In this embodiment, the three electrodes 200 are visible, however it may be appreciated that additional electrodes may be present around the expandable member 202. It may be appreciated that any number of electrodes 200 may be present, acting as a single electrode or acting independently or in combination. Placement of the electrodes 200 and/or selective energizing of the electrodes 200 may direct the energy toward particular target locations. In some embodiments, the electrodes 200 are comprised of flexible circuit pads or other materials attached to the expandable member 202 or formed into the expandable member 202. In some embodiments, the electrodes 200 are distributed radially around the circumference of the expandable member 202 and/or distributed longitudinally along the length of the expandable member 202. Such designs may facilitate improved deployment and retraction qualities, easing user operation and compatibility with introducer lumens.

In some embodiments, a fluid is delivered to the target blood vessel either before, during or after energy delivery. In some embodiments, the fluid includes one or more agents. Example agents include drugs, medications, chemotherapy agents, immunotherapy agents, micelles, liposomes, embolics, nanoparticles, drug-eluting particles, genes, plasmids, and proteins, to name a few. The fluid may be delivered by any suitable method, such as systemically, regionally or locally, such as by injection through a separate device or through the catheter 102. Typically, the agent is able to bathe the target tissue and optionally dwell for biodistribution. In some embodiments, the effect of the energy delivered to the target blood vessel is enhanced by the presence of the fluid either before, during or after energy delivery. In other embodiments, the effect of the fluid delivered to the target blood vessel is enhanced by the delivery of the energy either before, during or after agent delivery.

FIGS. 8-12 illustrate example embodiments of catheters 102 that deliver a fluid and optionally one or more agents through or near the energy delivery body 108. In particular. FIG. 8 illustrates an embodiment of the catheter 102 of FIG. 2 configured to deliver fluid having one or more types of agents. Here the expandable member 202 is fillable with a fluid carrying one or more agents, such as through ports in the shaft 106). Likewise, the expandable member 202 is able to weep or pass (as indicated by arrows) the one or more agents 110 to the surrounding environment, such as through pores in the expandable member 202. It may be appreciated that in some embodiments a fluid, such as a conducting solution or saline solution, is passed through the expandable member 202 so as to act as a virtual electrode. Thus, energy delivered to the electrode 200 pads is transferred to the fluid and is conducted to locations that the fluid has spread. This increases the size, shape and characteristics of the delivery electrode. It may be appreciated that a conductive solution may be used alone or in combination with one or more agents 110.

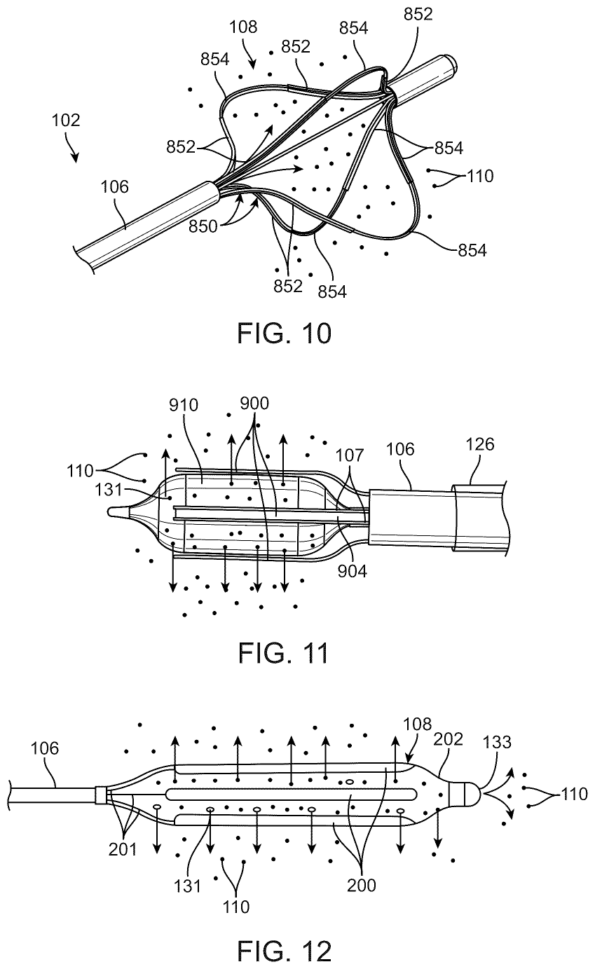

FIG. 9 illustrates an embodiment of the catheter 102 of FIG. 3 configured to deliver fluid, such as having one or more types of agents 110. In this embodiment, the shaft 106 includes a plurality of ports 131 and optionally a port 133 at the distal tip of the catheter 102 through which the agent 110 flows. Since the energy delivery body 108 comprises a wire basket, the agent 110 is able to go through the mesh of the basket to the surrounding environment. FIG. 10 illustrates an embodiment of the catheter 102 of FIG. 4 configured to deliver one or more types of agents. In this embodiment, the agent 110 is deliverable through a lumen in the shaft 106 so as to flow out into the area of the protrusions 850 as indicated by arrows. Thus, the agent 110 is able to freely pass into the surrounding environment of the energy delivery device 108. FIG. 11 illustrates an embodiment of the catheter 102 of FIGS. 6A-6B configured to deliver one or more types of agents. Here, the expandable member 910 includes a plurality of pores or ports 131 for passage of agent 110 therethrough, as indicated by arrows, to the surrounding environment. In this embodiment, the expandable member 910 is expanded (e.g. inflated) until the desired interface between the prongs 900 and vessel wall is achieved in coordination with desired flow rate of agent 110 through the ports 131. FIG. 12 illustrates an embodiment of the catheter 102 of FIG. 7 configured to deliver one or more types of agents. Here, the expandable member 202 includes a plurality of pores or ports 131 for passage of agent 110 therethrough, as indicated by arrows, to the surrounding environment. The ports 131 are located between the electrode 200 pads. Optionally, the catheter 102 includes a port 133 at the distal tip of the catheter 102 through which agent 110 flows. It may be appreciated that in each of the above embodiments, a fluid, such a conducting solution, may be used alone or in combination with the one or more agents 110.

### Clinical Methods

The therapeutic systems and devices may be used to treat a variety of conditions, particularly conditions of the vascular system such as atherosclerosis and angina. As mentioned previously, there are multiple types of angina including stable angina (angina pectoris), unstable angina, variant angina and microvascular angina. Both variant angina and microvascular angina involve spasm in the coronary arteries which supply blood to the heart muscle. During coronary spasm, the coronary arteries constrict or spasm on and off, causing temporary lack of blood supply to the heart muscle (ischemia). To diagnose coronary spasm, the patient may wear an ambulatory monitor, such as for up to 48 hours. The monitor records the electrical impulses of the heart, even during sleep. Changes on the electrocardiogram (EKG) may indicate coronary spasm. However, not all patients show EKG changes during every episode. To diagnose coronary spasm, an ergonovine stress test may be used. Ergonovine is a drug that is injected through an IV, usually during a cardiac catheterization. It can trigger coronary spasm, usually within minutes, at which point the coronary arteries are visualized. Another medication is then injected into the coronary artery to relieve the spasm. The patient's EKG is recorded before, during and after the test. If there is a coronary spasm, it can be seen on the EKG as well as an angiogram. It may be appreciated that other similar tests may also be used. In some instances, acetylcholine is used to trigger coronary spasm. Ergonovine acts through the serotogenic receptors, while acetylcholine acts through the muscarinic cholinergic receptors. Different mediators may have the potential to cause different coronary responses. Acetylcholine is supersensitive for females; spasm provoked by ergonovine is focal and proximal, whereas provoked spasm by acetylcholine is diffuse and distal. Therefore, both tests may be used as supplementary in the clinic.

Once the target tissue areas have been identified as associated with coronary spasm, the target tissue areas are treated with PEF energy. Optionally, one or more agents can be delivered as well in conjunction with the treatment. Typically, the catheter 102 is advanced through the vasculature to the coronary arteries and the energy delivery body 108 is positioned at the desired location. In some embodiments, the energy delivery body 108 is actuated so as to expand within the blood vessel and contact at least a portion of the blood vessel wall. PEF energy is delivered through the energy delivery body according to an algorithm as will be described in a later section. Typically, the PEF energy is delivered in a monopolar fashion. Monopolar delivery involves the passage of current from the energy delivery body 108 to the target tissue and through the patient to a return pad 140 positioned against the skin of the patient to complete the electric current circuit. Thus, in some embodiments, the catheter 102 includes only one energy delivery body 108 or electrode. This allows the catheter 102 to have a low profile so as to be positionable within smaller body lumens. This also allows deep penetration of tissue surrounding the energy delivery body 108. It may be appreciated that in some embodiments energy is delivered in a bipolar fashion, optionally with the use of more than one energy delivery body 108, however in some instances the monopolar delivery design simplifies the device and treatment design and provides superior treatment zones in target tissue.

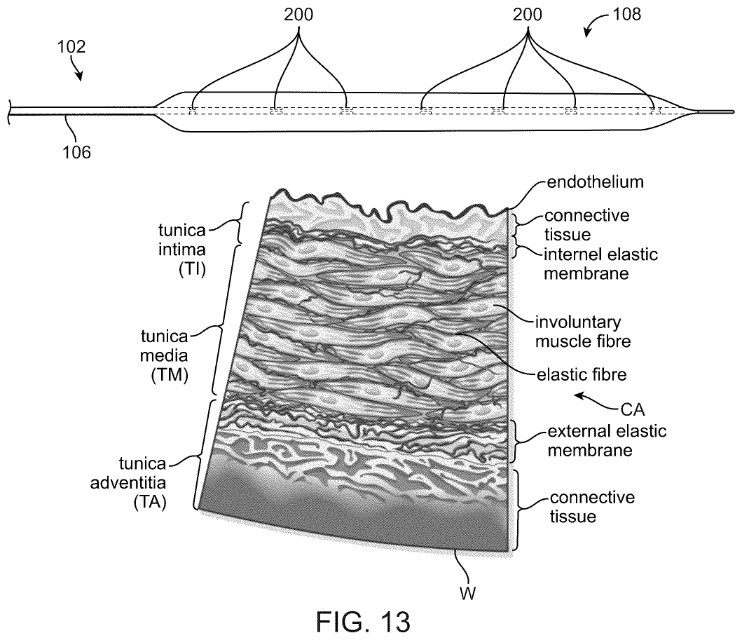

FIG. 13 includes a schematic illustration of a wall W of a coronary artery CA and an embodiment of an energy delivery body 108 (of FIG. 2) positioned adjacent the wall W. In this embodiment, the expandable member 202 is positioned against the endothelium E and the PEF energy is delivered from the electrodes 200 (as indicated by wavy lines) through the expandable member 202 to the wall W. In some embodiments, the PEF energy treats cells within the tunica intima TI (e.g. endothelium, connective tissue, internal elastic membrane, etc.) and/or tunica media TM (e.g. elastic fibers, smooth muscle cells, fibroblasts, extracellular matrix, etc.) and preserves the tunica adventitia TA (e.g. external elastic membrane, connective tissue, etc). The mechanisms proposed to constitute the basis for susceptibility to coronary artery spasm include endothelial dysfunction, a primary hyperreactivity of vascular smooth muscle cells (VSMCs), and other factors. Arterial spasm typically results from the interaction of at least two components (1) a localized, but sometimes diffuse, abnormality of an artery that causes hyperreactivity to vasoconstrictor stimuli, and (2) a vasoconstrictor stimulus able to induce the spasm at the level of the hyperreactive vascular segment. In some embodiments, delivery of PEF energy interferes with one or more of these components. For example, in some instances, the PEF energy treats the vascular smooth muscle cells which decreases or eliminates the ability of the blood vessel to vasoconstrict in response to stimuli. The extracellular matrix scaffold remains unaffected and preserves blood vessel patency as well as provides the proper environment for regeneration of the vascular wall. In some instances, the PEF energy disrupts the local innervation to the vascular smooth muscle cells which decreases or eliminates the local triggering stimuli for vascular spasm. In some embodiments, local denervation eliminates the neural pathway from the local area of spasm through the vagal afferent fibers that contribute to anginal pain symptoms felt in the chest, neck and jaw. In other embodiments, the PEF energy treats the endothelium leading to cell regeneration and healthy modification of the endothelial layer. In yet other embodiments, the PEF energy is utilized to modify the local cardiac myocardium; resulting in replacement or elimination of regional mechanical forces that may potentiate or precipitate spasm. It may be appreciated that the PEF energy may provide any combination or sub-combination of these effects.

It may be appreciated that treatment may include cell death, cell removal, and cell modification, to name a few. The tunica adventitia is the outermost layer of the coronary artery CA and is the strongest of the three layers. It is comprised of collagenous and elastic fibres. The tunica adventitia provides a limiting barrier, protecting the vessel from overexpansion. This layer is preserved to preserve the integrity of the vessel.

The tunica media TM varies based on the type of blood vessel being treated. In the smaller arteries it consists principally of smooth muscle fibers in fine bundles, arranged in lamella and disposed circularly around the vessel. These lamella vary in number according to the size of the vessel; the smallest arteries having only a single layer and those slightly larger have three or four layers, up to a maximum of six layers. It is this coat that largely determines the thickness of the wall of the artery. In the larger arteries, as the iliac, femoral, and carotid, elastic fibers and collagen unite to form lamella which alternate with the layers of smooth muscular fibers; these lamella are united to one another by elastic fibers which pass between the smooth muscular bundles, and are connected with the fenestrated membrane of the inner coat. In the largest arteries, as the aorta and brachiocephalic, the amount of elastic tissue is considerable; in these vessels a few bundles of white connective tissue also have been found in the middle coat. The muscle fiber cells are arranged in 5 to 7 layers of circular and longitudinal smooth muscle and contain well-marked, rod-shaped nuclei, which are often slightly curved. It may be appreciated the devices, systems and methods described herein are not limited to the treatment of coronary arteries and include treatment of intracranial arteries (e.g. cerebral artery spasm), peripheral arteries (e.g. superficial femoral, popliteal, tibial arteries) and other peripheral targets, to name a few. Likewise, anatomical targets include affected blood vessel segment(s) and/or adjacent segments, venous structure(s) adjacent to an affected blood vessel segment and/or adjacent segments, neural tissue adjacent to or controlling the affected blood vessel segment and/or adjacent segments, and muscular tissue adjacent to or controlling the affected blood vessel segment and/or adjacent segments (e.g., myocardium), to name a few. Example devices and systems for delivering energy to myocardium are provided in international patent application number PCT/US2020/066205 filed on Dec. 18, 2020, entitled “TREATMENT OF CARDIAC TISSUE WITH PULSED ELECTRIC FIELDS”, incorporated herein by reference for all purposes.

The therapeutic systems and devices may also be used to treat atherosclerosis or coronary artery disease, particularly restenosis after percutaneous transluminal angioplasty (PTA) and/or stent implantation. PTA uses dilation of a balloon at the end of a catheter at the stenosis to increase the lumen of the blood vessel. However, the increase in diameter can result in denudation of the endothelium, disruption of the internal elastic membrane and the tunica media, and damage to about 20% of the smooth muscle cells in the tunica media. Stents are designed to be expanded within a stenotic area in order to hold it open, using PTA either during or prior to application in order to expand the vessel with the stent. However, stents also lead to a disruption of normal vasculature. The use of PTA to expand the vessel wall with the stent will have similar effects to those previously described; and self-expanding stents can continue expanding due to radial forces, prolonging disturbances to endothelial function.

Changes to vessel architecture and cells from PTA and stenting can lead to the development of restenosis, a re-narrowing of the vessel at the site of intervention due to the iatrogenic injury response of the blood vessel. This problem also exists in the peripheral vasculature. Restenosis involves two major processes, arterial remodeling and neointimal hyperplasia. Arterial remodeling is a natural compensatory response, where the arteries enlarge in response to plaque formation to reduce vessel narrowing. However, in response to angioplasty, negative remodeling can lead to vessel constriction, reducing the overall vessel lumen. This is believed to be a primary mechanism for angioplasty restenosis, while in-stent restenosis appears to result primarily from neointimal hyperplasia, where vessel injury releases mitogens, and levels of mitogenic proto-oncogenes in the smooth muscle cells increase, altering smooth muscle cell phenotype, from contractile to synthetic, and 20 to 40% of medial smooth muscle cells enter the cell cycle within 3 days. Pro-migratory proteins are also expressed, promoting medial smooth muscle cells to migrate into the intima. Damaged endothelial cells from PTA and stent insertion may further contribute to smooth muscle cell proliferation and migration by decreasing their nitric oxide production, a chemical known to inhibit smooth muscle cell growth.

Target regions can be identified by any suitable methods, such as by computed tomography, angiography, intravascular ultrasound (IVUS), optical coherence tomography (OCT), or any suitable imaging modality. Once the target tissue areas have been identified, the target tissue areas are treated with PEF energy. In some instances, the PEF energy disrupts plaque layers, such as calcified deposits. In some embodiments, plaque is treated by other methods and the PEF energy is utilized such as to reduce or prevent restenosis. In some embodiments, one or more agents are delivered in conjunction with the PEF treatment. Typically, the catheter 102 is advanced through the vasculature to the coronary arteries (however other blood vessels may be targeted) and the energy delivery body 108 is positioned at the desired location. In some embodiments, the energy delivery body 108 is actuated so as to expand within the blood vessel and contact at least a portion of the blood vessel wall. PEF energy is delivered through the energy delivery body according to an algorithm as will be described in a later section. Typically, the PEF energy is delivered in a monopolar fashion. Monopolar delivery involves the passage of current from the energy delivery body 108 to the target tissue and through the patient to a return pad 140 positioned against the skin of the patient to complete the electric current circuit. Thus, in some embodiments, the catheter 102 includes only one energy delivery body 108 or electrode. It may be appreciated that in some embodiments energy is delivered in a bipolar fashion, optionally with the use of more than one energy delivery body 108.

In some embodiments, an agent 110 is also delivered to the target tissue area. Example agents include antiproliferative drugs which stop vascular smooth muscle cell proliferation and thus, neointimal hyperplasia. Sirolimus and paclitaxel are two examples of such drugs. Paclitaxel inhibits microtubule disassembly and thus interferes with the cell cycle, leading to cell cycle arrest in G0-G1 and G2-M phases. Sirolimus binds to the FKBP12 and subsequently inhibits mTOR and PI3 pathway, arresting the cell cycle in the G1 phase. Similar drugs have evolved from siroliumus and include everolimus (SDZ RAD) which has shown anti-arteriosclerotic features, ridaforolimus, zotarolimus, rapamycin agents, biolimus, and novolimus, etc. Other therapeutic agents 110 include antiproliferative drugs, anti-thrombotic drugs, phytoncide (PTC) which shows the same anti-inflammatory and antiproliferative effects in vitro as sirolimus and thus might serve as an alternative to sirolimus, glucocorticoids which are useful to suppress inflammatory changes that lead to restenosis, plasmid DNA to express appreciated proteins inside cells, galangin which up-regulates p27KIP1 that arrests cell cycle in the G0-G1 phase and inhibits proliferation of vascular smooth muscle cells, tacrolimus which reduces restenosis via the calcineurin/NFAT/IL-2 pathway, stem-cells to support healthy reendothelialization, radioactive agents, actinomycin, probucol, and 7-Hexanoyltaxol to name a few.

As mentioned, the agents 110 may be delivered by any suitable method, such as systemically, regionally or locally, such as by injection through a separate device or through the catheter 102. The agent 110 is able to bathe the target tissue and optionally dwell for biodistribution. In some embodiments, the PEF energy increases uptake of the agent 110 by the wall W of the blood vessel targeted by the PEF treatment. Thus, the effect of the agent 110 delivered to the target blood vessel is enhanced by the delivery of the energy either before, during or after agent delivery.

It may be appreciated that in some embodiments the PEF energy optionally removes, destroys, or kills cells within the wall of the blood vessel. Likewise, in some embodiments the PEF energy optionally disrupts calcified regions within the vessel, allowing better expansion. If calcium disruption is part of the therapy protocol, the catheter 102 or a separate device can be used after PEF delivery to hyperextend the vessel diameter. In some embodiments, local uptake of one or more agents 110 kills cells or inhibits hyperplastic regrowth of the cells in the target region of tissue. In some embodiments, the locally increased concentration of agent 110 that has been taken up will induce cell death in the affected region (the region exposed to electric field distributions), killing cells in the targeted region. Ultimately, tissue resolution and increased vascular flow occurs through the increased diameter lumens in the targeted region. The dead regions of tissue resolve and produce less physical restriction on lumen diameter and blood flow. And, the agents 110 slow or prevent regenerative hyperplastic regrowth over an extended period of time (e.g weeks to years).

### Energy Algorithms

The PEF energy is provided by one or more energy delivery algorithms 152. In some embodiments, the algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time or inter-phase delays between polarities in biphasic pulses, dead time or cycle delays between biphasic cycles, rest time or inter-packet delays between packets, or delays between groups or bundles of packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

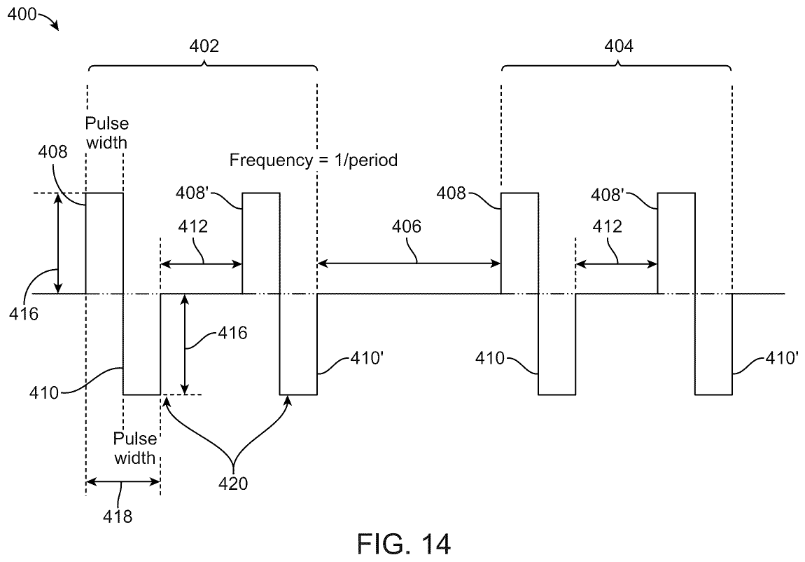

FIG. 14 illustrates an embodiment of a waveform 400 of a signal prescribed by an energy delivery algorithm 152. Here, two packets are shown, a first packet 402 and a second packet 404, wherein the packets 402, 404 are separated by a rest period 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time 412 (i.e., a pause) between each pulse. In this embodiment, the biphasic pulses are symmetric so that the set voltage 416 is the same for the positive and negative peaks. Here, the biphasic, symmetric waves are also square waves such that the magnitude and time of the positive voltage wave is approximately equal to the magnitude and time of the negative voltage wave. When using a bipolar configuration, portions of the cells facing the negative voltage wave undergo cellular depolarization in these regions, where a normally negatively charged cell membrane region briefly turns positive. Conversely, portions of the wall W cells facing the positive voltage wave undergo hyperpolarization in which the cell membrane region's electric potential becomes extremely negative. It may be appreciated that in each positive or negative phase of the biphasic pulse, portions of the wall W cells will experience the opposite effects. For example, portions of cell membranes facing the negative voltage will experience depolarization, while the portions 180° to this portion will experience hyperpolarization. In some embodiments, the hyperpolarized portion faces the dispersive or return electrode 140.

Desired treatment depths depend on the thickness of the wall W of the target vessel and on the type of treatment desired, to name a few. Vessels have a variety of wall thicknesses, such as approximately 2 mm (aorta), 1-4 mm (artery), 0.5-5 mm (vein), 1.5 mm (vena cava), 6 μm-30 μm (arteriole), 2 μm-10 μm (terminal arteriole). 0.5 μm-8 μm (capillary), 1 μm-20 μm (venule), etc. Likewise, particular layers within a vessel will vary in depth depending on the type of vessel. In addition, layers of calcification within blood vessels due to atherosclerosis may also increase the wall thickness, desiring deeper penetration depths than would be desired in an uncalcified vessel. Therefore, target treatment depths will vary. In some embodiments, the treatment depth into the wall W is in a range 0.5 μm-5 mm, particularly 0.5 μm-30 μm, more particularly 0.05 mm-5 mm, including 0.1 mm, 0.2 mm, 0.3 mm, 0.4 mm, 0.5 mm, 0.6 mm, 0.7 mm, 0.8 mm, 0.9 mm, 1 mm, 1.2 mm, 1.4 mm, 1.6 mm, 1.8 mm, 2 mm, 2.2 mm, 2.4 mm, 2.6 mm. 2.8 mm, 3 mm, 3.2 mm, 3.4 mm, 3.6 mm, 3.8 mm, 4.0 mm, 4.5 mm. In some embodiments, due to calcium deposits, treatment depths through the calcified plaque and into the wall W are in the range of 2-10 mm.

The energy delivered would depend on the desired treatment depth and on the type of treatment desired, to name a few. For example, treatments aimed at cell disruption or cell death would generally be weaker than treatments aimed at disruption of calcium deposits in arteriosclerosis. The following parameters may be used in a variety of treatment situations.

### A. Voltage

The voltages used and considered may be the tops of square-waveforms, may be the peaks in sinusoidal or sawtooth waveforms, may be the RMS voltage of sinusoidal or sawtooth waveforms or other suitable aspects. In some embodiments, the energy is delivered in a monopolar fashion and each high voltage pulse or the set voltage 416 is between about 100 V to 10,000 V. For cell death, voltages may typically be in a range of 500-3000V, particularly 2000-2500V. For disruption of calcium deposits, voltages may typically be in a range of 1000-6000V, particularly 2000-4000V. Voltages delivered to the tissue may be based on the setpoint on the generator 104 while either taking in to account the electrical losses along the length of the catheter 102 due to inherent impedance of the catheter 102 or not taking in to account the losses along the length, i.e., delivered voltages can be measured at the generator or at the tip of the catheter.

It may be appreciated that the set voltage 416 may vary depending on whether the energy is delivered in a monopolar or bipolar fashion. In bipolar delivery, a lower voltage may be used due to the smaller, more directed electric field. The bipolar voltage selected for use in therapy is dependent on the separation distance of the electrodes, whereas the monopolar electrode configurations that use one or more distant dispersive pad electrodes may be delivered with less consideration for exact placement of the catheter electrode and dispersive electrode placed on the body. In monopolar electrode embodiments, larger voltages are typically used due to the dispersive behavior of the delivered energy through the body to reach the dispersive electrode, on the order of 10 cm to 100 cm effective separation distance. Conversely, in bipolar electrode configurations, the relatively close active regions of the electrodes, on the order of 0.5 mm to 10 cm, including 1 mm to 1 cm, results in a greater influence on electrical energy concentration and effective dose delivered to the tissue from the separation distance. For instance, if the targeted voltage-to-distance ratio is 3000 V/cm to evoke the desired clinical effect at the appropriate tissue depth (1.3 mm), if the separation distance is changed from 1 mm to 1.2 mm, this would result in a necessary increase in treatment voltage from 300 to about 360 V, a change of 20%.

### B. Frequency

It may be appreciated that the number of biphasic cycles per second of time is the frequency when a signal is continuous. In some embodiments, biphasic pulses are utilized to reduce undesired muscle stimulation, particularly cardiac muscle stimulation. In other embodiments, the pulse waveform is monophasic and there is no clear inherent frequency. Instead, a fundamental frequency may be considered by doubling the monophasic pulse length to derive the frequency.

In some embodiments, the signal has a frequency in the range 10 kHz-800 kHz, For cell death, frequencies may typically be in a range of 100-800 kHz, more particularly 400-600 KHz. For disruption of calcium deposits, frequencies may typically be in a range of 10 μs-100 μs (monophasic) or 10-500 kHz (biphasic), more particularly 50-250 kHz. In some embodiments, the signal has a frequency in the range of approximately 100-600 kHz which typically penetrates the lumen wall so as to treat or affect particular cells somewhat deeply positioned, such as submucosal cells or smooth muscle cells. It may be appreciated that in some circumstances and at some voltages, frequencies at or below 100-250 kHz may cause undesired muscle stimulation. However, such muscle contraction may be mitigated by other techniques. Therefore, in some embodiments, the signal has a frequency in the range of 400-800 kHz or 500-800 kHz, such as 500 kHz, 550 kHz, 600 kHz, 650 kHz, 700 KHz, 750 kHz, 800 KHz. In particular, in some embodiments, the signal has a frequency of 600 kHz. In addition, cardiac synchronization may be utilized to reduce or avoid undesired cardiac muscle stimulation during sensitive rhythm periods. It may be appreciated that even higher frequencies may be used with components which minimize signal artifacts.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 800 kHz, while in other cases the treatment may be delivered using 2000 V with a waveform frequency of 400 KHz.

When used in opposing directions, the treatment parameters may be manipulated in a way that makes it too effective, which may increase muscle contraction likelihood or risk effects to undesirable tissues. For instance, if the frequency is increased and the voltage is decreased, such as the use of 2000 V at 800 kHz, the treatment may not have sufficient clinical therapeutic benefit. Opposingly, if the voltage was increased to 3000 V and frequency decreased to 400 KHz, there may be undesirable treatment effect extent to collateral sensitive tissues. In some cases, the over-treatment of these undesired tissues could result in morbidity or safety concerns for the patient. In other cases, the overtreatment of the untargeted or undesirable tissues may have benign clinical outcomes and not affect patient response or morbidity if they are overtreated.

### D. Packets

As mentioned, the algorithm 152 prescribes a signal having a waveform comprising a series of energy packets wherein each energy packet comprises a series of high voltage pulses. The cycle count 420 is half the number of pulses within each biphasic packet. Referring to FIG. 11, the first packet 402 has a cycle count 420 of two (i.e. four biphasic pulses). In some embodiments, the cycle count 420 is set between 1 and 100 per packet, including all values and subranges in between. In some embodiments, the cycle count 420 is up to 5 pulses, up to 10 pulses, up to 25 pulses, up to 40 pulses, up to 60 pulses, up to 80 pulses, up to 100 pulses, up to 1,000 pulses or up to 2,000 pulses, including all values and subranges in between. In some embodiments, cycle count is adjusted to achieve a desired total on-time of energy. For cell death, total on-time for a treatment may be 25-250 μs, more particularly 50-150 μs. For disruption of calcium deposits, total on-time for a treatment may be 50-500 μs, more particularly 75-150 μs.

The packet duration is determined by the cycle count, among other factors. Typically, the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

The number of packets delivered during treatment, or packet count may vary. In some embodiments, the number of packets per electrode activation are in a range of 1-30, more particularly 1-10. The number of packets delivered may be repeated or changed from one activated electrode to subsequent activated electrodes. This may be performed for monopolar and bipolar electrode arrangements.

### Example Parameter Combinations Include:

| | | Packet | Minimum # of | | |
|---|---|---|---|---|---|
| Voltage | Frequency | duration | Packets | Penetration | |
| 3500 V | 500 kHz | 250 μs | 200 | 1-10 | mm |
| 5000 V | 5 kHz | 200 μs | 10-20 | 5-20 | mm |
| 2500 V | 500 kHz | 100 μs | 50 | 5 | mm |
| 2500 V | 600 kHz | 100 | 20 | 0.5-1 | mm |
| 2500 V | 400 kHz | 100 μs | 5 | 0.5-2 | mm |
| 2000 V | 400 kHz | 100 μs | 10 | 0.5-2 | mm |
| 4000 V | 600 kHz | 100 μs | 10 | 0.5-2 | mm |
| 3000 V | 600 kHz | 100 μs | 5 | 0.5-1 | mm |
| 3000 V | 100 kHz | 100 μs | 10 | 1-5 | mm |
| 4000 V | 100 kHz | 100 μs | 10 | 2-6 | mm |
| 4000 V | 100 kHz | 100 μs | 20 | 4-15 | mm |
| 5000 V | 100 kHz | 100 μs | 20 | 10-20 | mm |

### E. Inter-Packet Delay

In some embodiments, the time between packets, referred to as the rest period 406 or inter-packet delay, is set between about 0.1 seconds and about 5 seconds, including all values and subranges in between. In other embodiments, the rest period 406 ranges from about 0.001 seconds to about 10 seconds, including all values and subranges in between. In some embodiments, the rest period 406 is approximately 1 second. In other embodiments, rest periods may reach 30 seconds, 1 min or 5 min. In particular, in some embodiments the signal is synced with the cardiac rhythm so that each packet is delivered synchronously within a designated period relative to the heartbeats, thus the rest periods coincide with the heartbeats. In other embodiments wherein cardiac synchronization is utilized, the rest period 406 may vary, as the rest period between the packets can be influenced by cardiac synchronization, as will be described in later sections.

### F. Batches

To ensure safety of treatment for cardiac rhythm, treatments may be delivered synchronously, whereby the PEFs are delivered in the safe S-T interval of the heart rhythm. Treatments may be delivered with multiple packets per heartbeat (faster, more potential thermal effects), or with multiple heartbeats between packets (slower, but reduces potential thermal effects). Similarly, whereby the biphasic waveform permits asynchronous delivery with minimal cardiac arrythmia risk, it is possible to deliver packets at a cadence that appropriately balances the time of treatment delivery (including consideration for adjuvant material bioavailability in the blood or locoregional space) with thermal load (temperature and time held at elevated temperatures that may affect the safety profile of the treatment).

In some embodiments, the signal is synced with the cardiac rhythm so that each packet is delivered synchronously within a designated period relative to the heartbeats, thus the rest periods coincide with the heartbeats. It may be appreciated that the packets that are delivered within each designated period relative to the heartbeats may be considered a batch or bundle. Thus, each batch has a desired number of packets so that at the end of a treatment period, the total desired number of packets have been delivered. Each batch may have the same number of packets, however in some embodiments, batches have varying numbers of packets.

In some embodiments, only one packet is delivered between heartbeats. In such instances, the rest period may be considered the same as the period between batches. However, when more than one packet is delivered between batches, the rest time is typically different than the period between batches. In such instances, the rest time is typically much smaller than the period between batches. In some embodiments, each batch includes 1-10 packets, 1-5 packets, 1-4 packets, 1-3 packets, 2-3 packets, 2 packets, 3 packets, 4 packets 5 packets, 5-10 packets, to name a few. In some embodiments, each batch has a period of 0.5 ms-1 sec, 1 ms-1 sec, 10 ms-1 sec. 10 ms-100 ms, to name a few. In some embodiments, the period between batches is variable, depending on the heart rate of the patient. In some instances, the period between batches is 0.25-5 seconds.

Treatment of a tissue area ensues until a desired number of batches are delivered to the tissue area. In some embodiments, 2-50) batches are delivered per treatment, wherein a treatment is considered treatment of a particular tissue area. In other embodiments, treatments include 5-40 batches, 5-30) batches, 5-20 batches, 5-10 batches, 5 batches, 6 batches, 7 batches, 8 batches, 9 batches, 10 batches, 10-15 batches, etc.

### G. Phase Delay and Inter-Cycle Delay

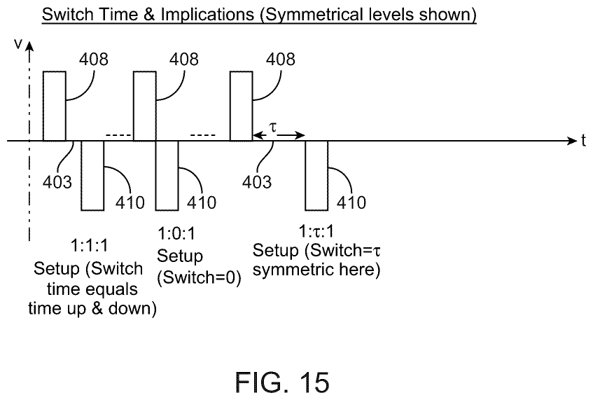

A switch time or phase delay is a delay or period of no energy that is delivered between the positive and negative peaks of a biphasic pulse, as illustrated in FIG. 15. FIG. 15 illustrates various examples of biphasic pulses (comprising a positive peak 408 and a negative peak 410) having a switch time 403 therebetween (however when the switch time 403 is zero, it does not appear). In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microseconds, including all values and subranges in between.

Delays may also be interjected between each cycle of the biphasic pulses, referred as an inter-cycle delay or “dead-time”. Dead time occurs within a packet, but between biphasic pulses. This is in contrast to rest periods which occur between packets. In other embodiments, the dead time 412 is in a range of approximately 0 to 0.5 microseconds, 0 to 10 microseconds, 2 to 5 microseconds, 0 to 20 microseconds, about 0 to about 100 microseconds, or about 0 to about 100 milliseconds, including all values and subranges in between. In some embodiments, the dead time 412 is in the range of 0.2 to 0.3 microseconds. Dead time may also be used to define a period between separate, monophasic, pulses within a packet.

Some PEF waveforms induce strong pressure waves and potential electric arcing events. Examples of such waveforms include single-pulse PEF waveforms or stacked cycle PEF waveforms (back-to-back cycles with >50% duty cycle) durations >roughly 10 μs (current-dependent). This can occur even into well-connected and high conductivity solutions. This typically occurs when the current density is too high due to concentration at singular points or small electrode. Both effects can induce severe adverse events for patients, and thus treatments generally must be titrated to intensities below those which induce these phenomena. However, it may be appreciated that in some instances a particular level of pressure waves and external arcing effects may be tolerated or even beneficial. For example, when disrupting calcium deposits in the treatment of atherosclerosis the external discharge may deliver a pressure wave similar to histotripsy that could mechanically break up the calcium deposits. Thus, in some embodiments, phase delays or inter-cycle delays may be short, minimal or zero. In some embodiments, this would support lower frequencies (<100 kHz, thru to monophasic waveforms up to 100 μs or 1 ms or 10 ms long). In some embodiments, asymmetric waveforms thru to monophasic waveforms may be used pressure wave generation.

In some embodiments, safe PEF waveforms are provided that include strategically timed energy delivery, by breaking packets into smaller sub-components of very short duration and with meaningfully small duty cycles. This is achieved with the introduction of specifically placed and timed delays, such as inter-pulse delays 14, inter-cycle delays 16, inter-phase delays 18, inter-packet delays 22, inter-bundle delays 26, etc. It may be appreciated that a combination of delays may be utilized within a treatment to obtain a desired outcome. In particular, these delays may be specifically manipulated to obtain particular desired outcomes. For example, one, some or all of these delays may be manipulated to control various aspects of PEF therapy so as to mitigate any associated risks, such as gas formation, electrical discharge, cavity formation, muscle contraction, and temperature rise, to name a few. In some embodiments, the delays distribute the period over which (high) voltage PEF energy is delivered, resulting in marked changes and optimization to the treatment delivery outcomes. In some embodiments, the range of delays described herein are between 0 s and 100 ms.

In some embodiments, the delay periods are manipulated to distribute the pace of energy delivery and permit resolution and decay of certain effects prior to them inducing effects from their accumulation. When applying PEFs for biological cell and tissue manipulation, where charge accumulation and decay is at a different timescale than the other effects, it is possible to accumulate treatment effect on the cell with multiple cycles or series of pulses, but without causing a variety of secondary treatment effects, such as gas formation, electrical discharge, cavity formation, muscle contraction, and temperature rise, to name a few. In other instances, these secondary accumulated treatment effects may be desirable to initiate or enhance therapy outcomes, and thus the delays will be selected to encourage these effects, which again are done in a manner that does not alter the primary objective of inducing cellular and tissue responses to the PEFs. These examples of secondary effects are not an exhaustive list and other secondary effects desired to be manipulated may also be controlled by selecting appropriate delays. Example delays and relationships to secondary effects are provided in international patent application number PCT/US2021/026221 filed on Apr. 8, 2021, entitled “PULSED ELECTRIC FIELD WAVEFORM MANIPULATION AND USE”, incorporated herein by reference for all purposes.

Overall, the susceptibility and sensitivity for a given therapy to each secondary treatment effect, such as gas formation, electrical discharge, cavity formation, muscle contraction, and temperature rise, will vary. Table 1, below, summarizes the potential most applicable ranges of delays that may be used to mitigate these effects for various targeted tissue varieties. Notably, this table focuses on applications to mitigate the secondary effects, but there are other times when these effects may want to be encouraged, and thus a different range of delays may be applicable for a given therapeutic target.

TABLE 1
Summary Table of Basic Cycle Delays

| | | | Cycle Delay |
|---|---|---|---|
| | Electrode(s) | | General Range |
| Target | Location | Objective | (Targeted Range) |
| Vascular | In blood, | Eliminate gas formation | 100 μs-10 ms |
| | adjacent to | | (250-1000 μs) |
| | target tissue | Eliminate electric discharge | 50 μs-10 ms |
| | | | (250-1000 μs) |
| | | Increase treatment effect | 100 μs-10 ms |
| | | | (500 μs-5 ms) |
| | | Reduce peak temperatures | 200 μs-20 ms |
| | | | (500 μs-10 ms) |
| | | Reduce muscle contraction | 5 ms-100 ms |
| | | | (10-30 ms) |

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for treating a blood vessel comprising: an energy delivery catheter comprising an energy delivery body positionable within or near the blood vessel; and a generator in electrical communication with the energy delivery body, wherein the generator includes at least one energy delivery algorithm that provides pulsed electric field energy to the energy delivery body so that the energy treats cells of the blood vessel while maintaining an extracellular matrix of the blood vessel in a manner that decreases or eliminates the ability of the blood vessel to vasconstrict.

2. A system as in claim 1, wherein treating comprises removing, destroying or killing the cells.

3. A system as in claim 2, wherein the cells comprise vascular smooth muscle cells involved in vasoconstriction of the blood vessel.

4. A system as in claim 1, wherein treating comprises disrupting local innervation to vascular smooth muscle cells.

5. A system as in claim 4, wherein disrupting local innervation comprises disrupting a neural pathway from a local area of spasm through at least one vagal afferent fiber that contributes to anginal pain symptoms.

6. A system as in claim 1, wherein the cells comprise endothelial cells and maintaining the extracellular layer leads to regeneration of an endothelial layer of the blood vessel.

7. A system as in claim 1, wherein the algorithm is configured to trigger delivery of the energy and a fluid in a timing sequence so that the fluid acts as a virtual electrode.

8. A system as in claim 7, wherein treating comprises causing the cells to uptake an agent.

9. A system as in claim 8, wherein the agent comprises a chemical, drug, medication, chemotherapy agent, immunotherapy agent, micelle, liposome, embolic, nanoparticle, drug-eluting particle, gene, plasmid, protein or combination of these.

10. A system as in claim 8, wherein uptake the agent inhibits hyperplastic regrowth of the cells.

11. A system as in claim 1, wherein the energy delivery body comprises an expandable member surrounding at least one electrode.

12. A system as in claim 11, wherein the energy delivery body comprises a shaft, wherein the at least electrode comprises a plurality of electrodes disposed along the shaft, and wherein the expandable member comprises an elongate expandable member surrounding the plurality of electrodes.

13. A system as in claim 12, wherein the expandable member is configured to weep fluid.

14. A system as in claim 1, wherein the energy delivery body comprises an electrode formed by a plurality of ribbons or wires.

15. A system as in claim 1, wherein the energy delivery body comprises one or more prongs, wherein each prong bends radially outward from a longitudinal axis of the energy delivery catheter.

16. A system as in claim 1, wherein the pulsed electric field energy is generated from a waveform including one or more of the following parameters a) a voltage in a range of 500-3000 V, b) a frequency in a range of 100-800 kHz, c) a total on-time in a range of 25-250 ps, and d) packets per electrode activation are in a range of 1-30 packets.

17. A system as in claim 1, wherein the pulsed electric field energy is generated from a waveform including one or more of the following parameters a) a voltage in a range of 1000-6000V b) a frequency in a range of 10-500 KHz c) a total on-time in a range of 50-500 ps, and d) packets per electrode activation are in a range of 1-30 packets.

18. A system for treating a vascular spasm comprising: an energy delivery catheter comprising an energy delivery body positionable on or near muscular tissue; and a generator in electrical communication with the energy delivery body, wherein the generator includes at least one energy delivery algorithm that provides pulsed electric field energy to the energy delivery body so that the energy modifies the muscular tissue so as to replace or eliminate regional mechanical forces that cause the vascular spasm.

19. A system as in claim 18, wherein the pulsed electric field energy is generated from a waveform including one or more of the following parameters a) a voltage in a range of 500-3000 V, b) a frequency in a range of 100-800 kHz, c) a total on-time in a range of 25-250 ps, and d) packets per electrode activation are in a range of 1-30 packets.

20. A system as in claim 18, wherein the pulsed electric field energy is generated from a waveform including one or more of the following parameters a) a voltage in a range of 1000-6000V b) a frequency in a range of 10-500 KHz c) a total on-time in a range of 50-500 ps, and d) packets per electrode activation are in a range of 1-30 packets.

21. A method treating a blood vessel comprising: positioning an energy delivery body of an energy delivery catheter within or near the blood vessel; and delivering pulsed electric field energy to the energy delivery body so that the energy treats cells of the blood vessel while maintaining an extracellular matrix of the blood vessel in a manner that decreases or eliminates the ability of the blood vessel to vasconstrict.

22. A method for treating a vascular spasm comprising: positioning an energy delivery body of an energy delivery catheter on or near cardiac myocardium; and delivering pulsed electric field energy to the energy delivery body so that the energy modifies the cardiac myocardium so as to replace or eliminate regional mechanical forces that cause the vascular spasm.
